(19) United States
(12) Patent Application Publication
(10) Pub. No.: US 2012/0060848 A1
(43) Pub. Date: Mar. 15, 2012

(54) ANTISNORING DEVICE

(76) Inventor: Bao Chien Chen, Taipei (TW)

(21) Appl. No.: 13/229,158

(22) Filed: Sep. 9, 2011

(30) Foreign Application Priority Data
Sep. 13, 2010 (TW) 99217670
Jan. 27, 2011 (TW) 100103012

(51) Int. Cl. A61F 5/56

(52) U.S. Cl. 128/848

(57) ABSTRACT

An anti-snoring device for occlusion between teeth on an upper jaw and a lower jaw is disclosed. The anti-snoring device of the present invention includes an upper tooth brace, a lower tooth brace and a connecting portion, wherein the upper and lower tooth braces have walls and tooth-bearing portions for positioning teeth on the upper and lower jaws, and the force is evenly applied on the teeth. The lower jaw is thus moved forward, and the throat soft tissues are prevented from moving downward. Thus, the trachea would not be pressed, and the respiratory tract would not be narrowed, such that air resistance in the respiratory tract would not be increased, and the vibration occurring in the respiratory tract would be decreased. Accordingly, the anti-snoring device of the present invention effectively prevents snoring.

### BACKGROUND OF THE INVENTION

1. Field of the Invention

The present invention relates to anti-snoring devices, and more particularly, to an anti-snoring device for occlusion between teeth on an upper jaw and a lower jaw in an oral cavity.

2. Description of Related Art

Snoring may be due to many reasons such as blockade of the respiratory tract, aging, obesity and etc. The major reason for snoring is shown in FIG. 1. When the gravity pulls down the soft tissue 12 such as the soft palate and the root of tongue, the respiratory tract 11 is narrowed, and snoring is the vibration of respiratory tract 11 and the resulting sound due to obstructed air movement during breathing.

There have been some treatments for snoring during sleep. For example, it is well known to use an anti-snoring device for moving the lower jaw forward. As shown in FIG. 2, the anti-snoring device 2 is worn on the teeth 14 to move the lower jaw 13 forward, so as to move forward the tongue soft tissue 12 such as the soft jaw and the root of the tongue. The tongue soft tissue 12 is thus prevented from moving down to the respiratory tract 11, so as to enable air to flow unobstructed through the breathing passage for stopping snoring. FIG. 3 is a schematic view showing the conventional anti-snoring device 2. As shown in FIG. 2, the anti-snoring device 2 includes an upper tooth brace 21 and a lower tooth brace 22, which are made of a hard material. The upper and lower tooth braces have recess portions 212, 222 to fit the shape of a user's teeth and reduce a gap between the anti-snoring device 2 and teeth 14, and have adhesive pads 23 at the right and left sides for connecting the upper and lower tooth braces 21, 22. Further, the upper tooth brace 21 has positioning portions 211 made of a metal for propping the user's gum.

However, the recess portions 212, 222 are formed via a mold of the user's teeth for reducing the gap between the anti-snoring device 2 and the teeth 14, and the upper and lower tooth braces 21, 22 are connected via the adhesive pads 23 to form the anti-snoring device 2. Hence, the anti-snoring device 2 has high fabrication cost, and thus cannot be used widely.

In addition, the positioning portions 211 are formed from stainless steel lines for minimization, but the gum propped by the positioning portions 211 makes the user uncomfortable and even causes inflammation to the gums.

Accordingly, it is an urgent issue to provide an anti-snoring device, which has low cost and provides comfort to users.

### SUMMARY OF THE INVENTION

The present invention provides an anti-snoring device, which has low cost and provides comfort to users.

The anti-snoring device of the present invention includes an upper tooth brace at least comprising a first tooth-bearing portion, a first inner wall, and a first outer wall, wherein a height of the first outer wall is 5 to 10 mm, and a height of the first inner wall is smaller than the height of the first outer wall; a lower tooth brace at least comprising a second tooth-bearing portion, a second inner wall and a second outer wall, wherein a height of the second inner wall is 6 to 10 mm, a height of the second outer wall is smaller than the height of the second inner wall, and a distance between the first outer wall and the second outer wall is 5 to 9 mm; and a connecting portion for connecting the upper tooth brace and the lower tooth brace and forming a space between a first tooth-bearing surface of the first tooth-bearing portion and a second tooth-bearing surface of the second tooth-bearing portion, wherein the space between the first tooth-bearing surface and the second tooth-bearing surface is gradually decreased from middle parts of the first and the second tooth-bearing portions to side ends of the first and the second tooth-bearing portions, and the connecting portion has an opening between the first and the second tooth-bearing portions, wherein the upper tooth brace, the lower tooth brace and the connecting portion are formed integrally, a space between the middle parts of the first and the second tooth-bearing portions is 12 to 20 mm, and a space between the side ends of the first and the second tooth-bearing portions is 4 to 8 mm.

The upper tooth brace and the lower tooth brace may respectively have gaps at two sides of the opening. The upper tooth brace, the lower tooth brace and the connecting portion are made of a rubber, a resin or a plastic. The shape of the opening is a rectangle, an oval or a circle. The angle between the first tooth-bearing surface and the second tooth-bearing surface is 8 to 15 degrees. The height of each of the inner walls and the outer walls is gradually decreased from the middle part to the side ends of each of the first and the second tooth-bearing portions.

The anti-snoring may further include a supporting portion around the opening. The anti-snoring further incudes a recess portion at a middle part of the first outer wall or the second inner wall. The space between the middle parts of the first and the second tooth-bearing portions is 13 to 17 mm, and the space between the side ends of the first and the second tooth-bearing portions is 5 to 7 mm. The height of the first inner wall is 2 to 5 mm, and the height of the second outer wall is 5 to 8 mm.

Therefore, the anti-snoring device includes an upper tooth brace, a lower tooth brace and a connecting portion, wherein the upper tooth brace and the lower tooth brace have walls and tooth-bearing portions for positioning upper jaw teeth and lower jaw teeth, moving the lower jaw forward and preventing soft tongue tissues from moving down. Hence, the respiratory tract would not be narrowed, so as to effectively avoid snoring. The anti-snoring device is formed integrally, such that the procedure of connecting the upper tooth brace and the lower tooth brace is omitted. Further, the anti-snoring device may be elastically deformed upon being worn by a user, such that there is no need to form a teeth mold for a user. Accordingly, the anti-snoring device has low cost and provides comfort to users.

### BRIEFE DESCRIPTION OF THE DRAWINGS

FIG. 1 is a schematic view showing a human's throat while snoring;

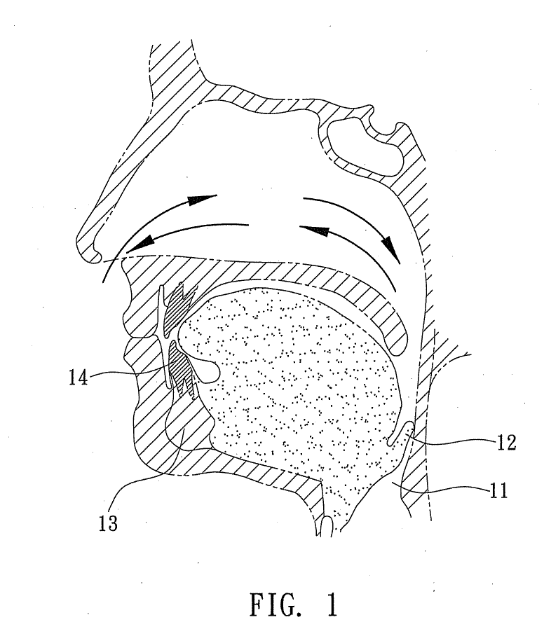

FIG. 2 is a schematic view showing a human's throat while the user is wearing the anti-snoring device;

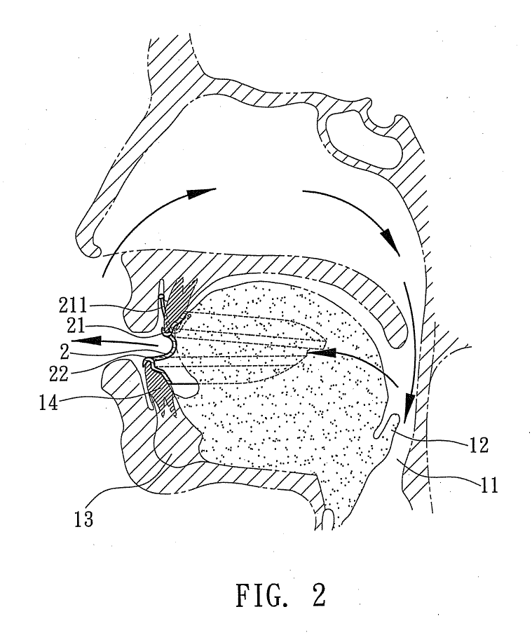

FIG. 3 is a schematic view showing the conventional anti-snoring device;

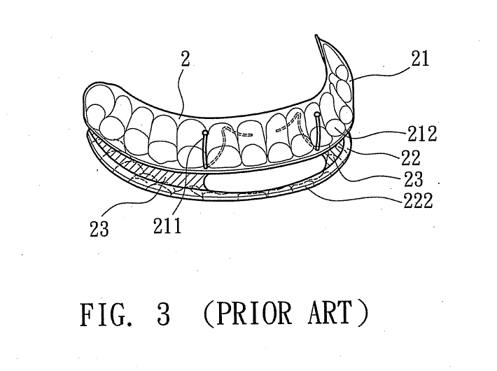

FIG. 4 is a schematic view showing an anti-snoring device according to an embodiment of the present invention;

FIG. 5 is a schematic view showing a user wearing the anti-snoring device of FIG. 4; and

FIG. 6 is a schematic view showing a user's throat while wearing the anti-snoring device of FIG. 4.

### DETAILED DESCRIPTION OF THE PREFERRED EMBODIMENTS

The detailed description of the present invention is illustrated by the following specific examples. Persons skilled in the art can conceive the other advantages and effects of the present invention based on the disclosure contained in the specification of the present invention.

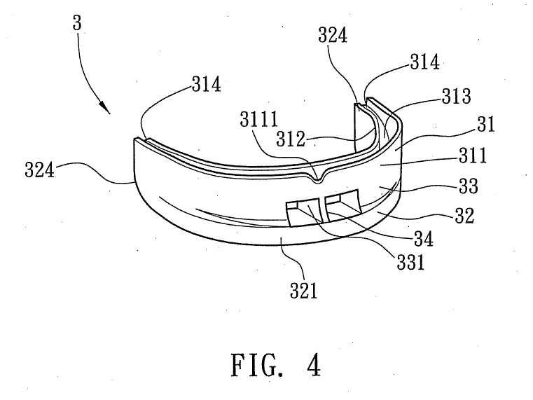

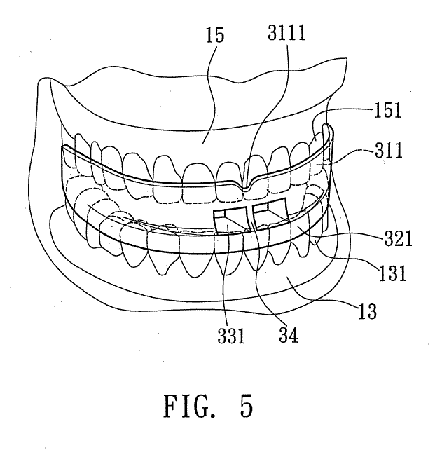

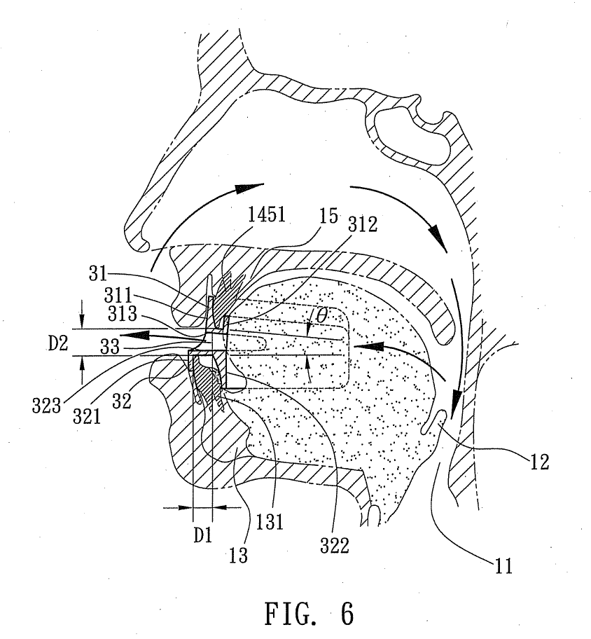

FIG. 4 is a schematic view showing an anti-snoring device according to an embodiment of the present invention; FIG. 5 is a schematic view showing a user wearing the anti-snoring device of FIG. 4; and FIG. 6 is a schematic view showing a user's throat while wearing the anti-snoring device of FIG. 4.

The anti-snoring device 3 is used for positioning teeth 151, 131 of the upper jaw 15 and the lower jaw 13. The anti-snoring device 3 includes an upper tooth brace 31, a lower tooth brace 32 and a connecting portion 33. The upper tooth brace 31, the lower tooth brace 32 and the connecting portion 33 are made of an elastic material such as a rubber, a resin or a plastic. Thus, while a user wears the anti-snoring device 3, the upper tooth brace 31 an the lower tooth brace 32 deform in response to the tooth-bearing force, so as to reduce the gap between the anti-snoring device 3 and the teeth and increase comfort to the user. Hence, there is no need to form a teeth mold for the user, so as to simplify the fabrication process, and reduce the production cost of the anti-snoring device 3.

Further, the upper tooth brace 31, the lower tooth brace 32 and the connecting portion 33 are formed integrally, such that the procedure of connecting the upper tooth brace and the lower tooth brace is omitted, and the anti-snoring device 3 thus has low production cost.

The upper tooth brace 31 includes a first tooth-bearing portion 313, a first inner wall 312 and a first outer wall 311. As shown in figures, the walls 312, 311 are sheet structures having a surface (for example, U-shaped surface) corresponding to the shape of the teeth on the upper jaw 15, such that the force is evenly applied on the teeth 151 on the upper jaw 15, so as to provide comfort to the user. The first tooth-bearing portion 313 has a tooth-bearing surface for the teeth 151 on the upper jaw 15 to occlude (referring to FIG. 5 and FIG. 6). The height of the first outer wall 311 is more than or equal to the half-height of the tooth crown of the front teeth on the upper jaw 15, so as to provide an enough area for propping the outer surface of the teeth 151 on the upper jaw 15 toward inside the oral cavity. Generally, the height of the tooth crown of the front teeth on the upper jaw is about 8 to 9 mm, and thus the height of the first outer wall 311 is about 5 to 10 mm. Preferably, thus the height of the first outer wall 311 is 7 to 9 mm. The height of the first inner wall 312 is smaller than the height of the first outer wall 311 (for example, small than the half-height of the tooth crown of the front teeth on the upper jaw 15), so as to provide an enough area for propping the inner surface of the front teeth on the upper jaw and to cooperate with the first outer wall 311 for preventing occlusion of the teeth 151 and the first tooth-bearing portion 313. The height of the first inner wall 312 is about 2 to 5 mm, and preferably 3 to 4.5 mm.

In this embodiment, the first inner wall 312 and the first outer wall 311 extend toward the gum of the upper jaw 15, so as to effectively prop the surface of the teeth 151 on the upper jaw 15. The upper tooth brace 31 covers the teeth 151 on the upper jaw 15, and the space between the first inner wall 312 and the first outer wall 311 is determined based on the thickness of the teeth on the upper jaw 15, and therefore the upper tooth brace 31 may cover the teeth 151 on the upper jaw 15. Further, a recess portion 3111 may be formed in the middle part of the outer wall 311 for facilitating the user to correctly wear the anti-snoring device 3. As shown in FIG. 6, the thickness of the first outer wall 311 may be gradually and downwardly increased according to the tooth shape on the upper jaw 15, so as to increase the structure strength of the first outer wall 311.

The lower tooth brace 32 includes a second tooth-bearing portion 323, a second inner wall 322 and a second outer wall 321. As shown in figures, the walls 322, 321 are sheet structures having a surface (for example, U-shaped surface) corresponding to the shape of the teeth on the lower jaw 13. The second tooth-bearing portion 323 has a tooth-bearing surface for the teeth 131 on the upper jaw 13 to occlude (referring to FIG. 5 and FIG. 6). The height of the second inner wall 322 is more than or equal to the half-height of the tooth crown of the front teeth on the upper jaw 13, so as to provide an enough area for propping the inner surface of the front teeth on the lower jaw 13 toward outside the oral cavity while the user is supine. The lower jaw is moved forward relatively to the upper jaw 15, so as to move the soft tissues 12 forward and prevent the soft tissues 12 from moving down to the respiratory tract 11, such that the respiratory tract 11 would not be narrowed. Thus, the air resistance in the respiratory tract 11 would not be increased, and the vibrations occurring in the respiratory tract 11 would be decreased. Accordingly, the anti-snoring device 3 of the present invention effectively prevents snoring. In this embodiment, the height of the second inner wall 322 is about 6 to 10 mm, and preferably 7 to 9 mm.

The height of the second outer wall 321 is smaller than the height of the second inner wall 322 (for example, smaller than half-height of the tooth crown of front teeth on the lower jaw 13), so as to provide an enough area for propping the outer surface of the front teeth on the lower jaw 13 and to cooperate with the second inner wall 322 for preventing occlusion between the teeth 131 on the lower jaw 13 and the second tooth-bearing portion 323. The height of the second outer wall 321 is 5 to 8 mm, and preferably 6 to 7 mm.

The second inner wall 322 and the second outer wall 321 extend toward the gum of the lower jaw 13, so as to effectively prop the surface of the teeth 131 on the lower jaw 13. The lower tooth brace 32 covers the teeth 131 on the lower jaw 13, and the space between the second inner wall 322 and the second outer wall 321 is determined based on the thickness of the teeth on the lower jaw 13, and therefore the lower tooth brace 32 may cover the teeth 131 on the lower jaw 13. As shown in FIG. 6, the thickness of the second inner wall 321 may be gradually and downwardly increased according to the tooth shape on the lower jaw 13, so as to increase the structure strength of the second inner wall 321 and increase comfort to a user wearing the anti-snoring device 3.

The connecting portion 33 is used for connecting the upper tooth brace 31 and the lower tooth brace 32. The space between the first outer wall 311 and the second outer wall 321 is D1, which is 1.5 to 2.5 folds of the thickness of the tooth crown of the front teeth on the upper jaw 15 and the lower jaw 13. Generally, the thickness of the tooth crown of the front teeth on the upper jaw 15 and the lower jaw 13 is about 3 to 4 mm, and thus the space between the first outer wall 311 and the second outer wall 321 is 5 to 9 mm. Preferably, the space between the first outer wall 311 and the second outer wall 321 is 5.5 to 7.5 mm. The connecting portion 33 is used for providing a space between the tooth-bearing surfaces of the first tooth-bearing portion 313 and the second tooth-bearing portion 323. The space between the tooth-bearing surfaces is gradually decreased from the middle parts to the side ends of the first and the second tooth-bearing potions 313, 323, such that there is an angle θ ranging from about 8 to 15 degrees (preferably 9 to 13 degrees) between the tooth-bearing surfaces of the first tooth-bearing portion 313 and the second tooth-bearing portion 323, and the lower jaw 13 is moved forward. Hence, the soft tissues 12 are prevented from moving down, so as to keep the respiratory tract 11 without obstruction and prevent snoring. In other words, the upper tooth brace 31 and the lower tooth brace 32 of the anti-snoring device 3 are used for positioning the teeth 151, 131, so as to move the lower jaw 13 forward and to prevent the lower jaw from moving backward. Thus, the trachea would not be pressed, and the respiratory tract 11 would not be narrowed.

The space between the middle parts of the first tooth-bearing portion 313 and the second tooth-bearing portion 323 is D2, which is 1.5 to 2 folds of the height of the tooth crown of the front teeth on the upper jaw 15 and the lower jaw 13. Generally, the height of the tooth crown of the front teeth on the upper jaw 15 is about 8 to 9 mm. Therefore, the space between the middle parts of the tooth-bearing surfaces of the first tooth-bearing portion 313 and the second tooth-bearing portion 323 is about 12 to 20 mm, and preferably 13 to 17 mm. In this embodiment, the space between the middle parts of the tooth-bearing surfaces of the first tooth-bearing portion 313 and the second tooth-bearing portion 323 is 4 to 8 mm, and preferably 5 to 7 mm.

The connecting portion 33 further includes an opening 331 between the upper tooth brace 31 and the lower tooth brace 32 for air to pass through the respiratory tract. Thus, the user has smooth breathing even while sleeping.

In this embodiment, the opening 331 is a rectangle. Certainly, the opening may be formed as an oval or a circle according to the breathing of the user. The anti-snoring device 3 may include a supporting portion 34 around the opening 331 for increasing the structure strength of the anti-snoring device 3.

In this embodiment, the walls 312, 311, 322, 321 of the tooth braces 31, 32 are used for respectively propping the inner surfaces and outer surfaces of the teeth 151, 131 on the upper jaw 15 and the lower jaw 13, such that the force is evenly applied on the teeth 151, 131, and the over pressure on local area is prevented. Hence, in comparison with the prior art, the anti-snoring device of the present invention provides more comfort to users.

Preferably, the first tooth-bearing portion 313, the first inner wall 312 and the first outer wall 311 of the upper tooth brace 31 extend to the position of the molar tooth on the upper jaw 15, and the second tooth-bearing portion 323, the second inner wall 322 and the second outer wall 321 of the lower tooth brace 32 extend to the position of the molar tooth on the lower jaw 13, such that the first tooth-bearing portion 313 and the second tooth-bearing portion 323 have enough occlusion areas, the first inner wall 312, the first outer wall 311, the second inner wall 322 and the second outer wall 321 have enough propping areas, and thus the upper tooth brace 31 and the lower tooth brace 32 cover the teeth 151, 131 on the upper jaw 15 and the lower jaw 13. A recess portion may be formed at the middle part of the second inner wall 322 for facilitating a user to wear the anti-snoring device 3.

As shown in FIG. 4, the upper tooth brace 31 and the lower tooth brace 32 respectively have gaps 312, 324 corresponding to two sides of the opening 3331 for the teeth 151, 131 on the upper jaw 15 and the lower jaw 13 to pass through, so as to facilitate the user to wear the anti-snoring device 3. The heights of the first inner wall 312 and the second inner wall 322 corresponding to one side of the opening 331 are gradually decreased according to the shapes of the upper jaw 15 and the lower jaw 13, so as not to press the upper jaw 15 and the lower jaw 13. The anti-snoring device 3 thus provides much more comfort to users.

Hence, the anti-snoring device of the present invention is used for occlusion between the teeth on the upper jaw and the lower jaw, and has an upper tooth brace, a lower tooth brace and a connecting portion, wherein the upper and lower tooth braces have walls and tooth-bearing portions for positioning teeth on the upper and lower jaws. The lower jaw is thus moved forward, and the throat soft tissues are prevented from moving downward. Thus, the trachea would not be pressed, and the respiratory tract would not be narrowed, such that air resistance in the respiratory tract would not be increased, and the vibration occurring in the respiratory tract would be decreased. Accordingly, the anti-snoring device of the present invention effectively prevents snoring.

Further, the upper tooth brace, the lower tooth brace and the connecting portion are made integrally of an elastic material, and thus the fabrication procedure of connecting the upper tooth brace and the lower tooth brace is omitted. Moreover, upon occlusion, the upper tooth brace, the lower tooth brace and the connecting portion may elastically deform, so as to effectively reduce the space between the anti-snoring device and the teeth. Therefore, there is no need to form a teeth mold according to a user's teeth. Hence, the anti-snoring device of the present invention has a simple fabrication procedure, and a large-scale of the anti-snoring device may be produced, such that the production cost of the anti-snoring device is low.

The walls of the upper tooth brace and the lower tooth brace have enough areas for propping teeth on the upper and lower jaws, so as to prevent over pressure on a local area on the gum, and to prevent a user from suffering uncomfortable.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

1. An anti-snoring device, comprising:
an upper tooth brace at least comprising a first tooth-bearing portion, a first inner wall, and a first outer wall, wherein a height of the first outer wall is 5 to 10 mm, and a height of the first inner wall is smaller than the height of the first outer wall;
a lower tooth brace at least comprising a second tooth-bearing portion, a second inner wall and a second outer wall, wherein a height of the second inner wall is 6 to 10 mm, a height of the second outer wall is smaller than the height of the second inner wall, and a distance between the first outer wall and the second outer wall is 5 to 9 mm; and
a connecting portion for connecting the upper tooth brace and the lower tooth brace and forming a space between a first tooth-bearing surface of the first tooth-bearing portion and a second tooth-bearing surface of the second tooth-bearing portion, wherein the space between the first tooth-bearing surface and the second tooth-bearing surface is gradually decreased from middle parts of the first and the second tooth-bearing portions to side ends of the first and the second tooth-bearing portions, and the connecting portion has an opening between the first and the second tooth-bearing portions,
wherein the upper tooth brace, the lower tooth brace and the connecting portion are formed integrally, a space between the middle parts of the first and the second tooth-bearing portions is 12 to 20 mm, and a space between the side ends of the first and the second tooth-bearing portions is 4 to 8 mm.

2. The anti-snoring device of claim 1, wherein the upper tooth brace and the lower tooth brace respectively have gaps at two sides of the opening.

3. The anti-snoring device of claim 1, wherein the upper tooth brace, the lower tooth brace and the connecting portion are made of a rubber, a resin or a plastic.

4. The anti-snoring device of claim 1, wherein a shape of the opening is a rectangle, an oval or a circle.

5. The anti-snoring device of claim 1, wherein an angle between the first tooth-bearing surface and the second tooth-bearing surface is 8 to 15 degrees.

6. The anti-snoring device of claim 1, wherein the height of each of the inner walls and the outer walls is gradually decreased from the middle part to the side ends of each of the first and the second tooth-bearing portions.

7. The anti-snoring device of claim 1, further comprising a supporting portion around the opening.

8. The anti-snoring device of claim 1, further comprising a recess portion at a middle part of the first outer wall or the second inner wall.

9. The anti-snoring device of claim 1, wherein the space between the middle parts of the first and the second tooth-bearing portions is 13 to 17 mm, and the space between the side ends of the first and the second tooth-bearing portions is 5 to 7 mm.

10. The anti-snoring device of claim 1, wherein the height of the first inner wall is 2 to 5 mm, and the height of the second outer wall is 5 to 8 mm.
